(19) United States
(12) Patent Application Publication
(10) Pub. No.: US 2006/0048288 A1
(43) Pub. Date: Mar. 9, 2006

(54) Eye goggles with unitary internal skeleton and overmoulded covering

(76) Inventor: Joseph Haslbeck, West Vancouver (CA)

Correspondence Address: KOLISCH HARTWELL, P.C., 200 PACIFIC BUILDING, 520 SW YAMHILL STREET, PORTLAND, OR, 97204, US

(21) Appl. No.: 10/930,189

(22) Filed: Aug. 30, 2004

(51) Int. Cl. A61F 9/02

(52) U.S. Cl. 2/439

(57) ABSTRACT

Eye goggles comprising an inner skeleton of a rigid material formed from a pair of transparent eye piece elements and an integral nose bridge element connected therebetween to permit relative movement between the eye piece elements and the nose bridge element. A covering partially covers the inner skeleton to define at least a pair of transparent regions in the eye piece elements. The eye goggles are simple and robust in structure and include an inner skeleton that acts to pre-shape the goggles and retain them in a desirable and comfortable configuration.

### FIELD OF THE INVENTION

The invention relates to eye goggles, in particular to eye goggles which can be used to protect the eyes in a wide variety of recreational activities, such as swimming, cycling, ball games, etc. where eye protection is usually worn.

### BACKGROUND OF THE INVENTION

Protective eye goggles for sports use are well known and serve to protect or isolate a wearer's eyes to prevent water, chemicals or other foreign materials from entering the eyes and obscuring vision. Many conventional goggle designs includes two rigid eyepieces which are interconnected by a flexible nosebridge piece, and a headband which passes around the wearer's head to hold the goggles on the face with the eyepieces positioned over the eyes to shield or even seal the eye sockets. Goggles of this type tend to be constructed from a plurality of individual parts (nose bridge portion, eyepieces, and headband) that must be manually assembled to create finished goggles. This is very labour intensive and creates a goggle structure that is prone to warping and breakage.

### SUMMARY OF THE INVENTION

To address these shortcomings, I have developed a novel eye goggle structure that is simple and robust in structure and includes an internal skeleton that acts to pre-shape the goggles and retain them in a desirable and comfortable configuration.

Accordingly, the present invention provides eye goggles comprising:

an inner skeleton of a rigid material comprising:

a pair of transparent eye piece elements; and

a nose bridge element formed integrally with the eye piece elements and connected therebetween to permit relative movement between the eye piece elements and the nose bridge element; and

a covering partially covering the inner skeleton to define at least a pair of transparent regions in the eye piece elements.

In a further aspect, the present invention provides a method of forming eye goggles comprising the steps of:

forming an inner skeleton of a rigid material having a pair of transparent eye piece elements and a nose bridge element formed integrally with the eye piece elements, and connected therebetween to permit relative movement between the eye piece elements and the nose bridge element; and

applying a covering partially covering the inner skeleton to leave at least a pair of transparent regions in the eye piece elements.

The construction of the present invention provides an eye goggle that maintains a desired pre-shaped configuration by virtue of its rigid inner skeleton for fitting over a wearer's eyes. While the eye goggles are sufficiently rigid to retain a desired extended shape for ease of fitting over the face and eyes, they are also comfortable due to the resilient covering. The goggles are also constructed from only two main component parts with the result that the overall structure is stronger and assembly costs are significantly reduced.

The goggles of the present invention also incorporate indicia for advertising or marking purposes comprising at least one opening in the outer covering to expose a region of the inner skeleton through the covering. The indicia are permanent and conspicuous, and are a significant improvement over the silkscreened or painted indicia of current goggle designs which are prone to wearing off.

### BRIEF DESCRIPTION OF THE DRAWINGS

Aspects of the present invention are illustrated, merely by way of example, in the accompanying drawings in which:

FIG. 1 is a front perspective view of a swimming goggle embodiment of the present invention;

FIG. 2 is a rear perspective view of the embodiment of FIG. 1;

FIG. 3 is a front elevation view of the embodiment of FIG. 1;

FIG. 4 is a top view of the embodiment of FIG. 1;

FIG. 5 is a section view taken along line 5-5 of FIG. 4 through an eye piece element of the embodiment;

FIG. 6 is a front perspective view of the inner skeleton of the swim goggle embodiment of the present invention prior to application of an overmoulded covering;

FIG. 7 is a rear perspective view of the inner skeleton of FIG. 6;

FIG. 8 is a front elevation view of the inner skeleton of FIG. 6;

FIG. 9 shows schematically a method for forming the indicia of the present invention using openings in the covering;

FIG. 10 shows schematically an alternative method for forming the indicia which relies on openings aligned with exposed protrusions from the inner skeleton;

FIG. 11 is a detail cross-section through the goggles of the present invention showing indicia formed by protrusions from both sides of the inner skeleton; and

FIG. 12 is a detail cross-section through the goggles of the present invention showing indicia formed by protrusions extending from only one side of the inner skeleton.

### DESCRIPTION OF THE PREFERRED EMBODIMENTS

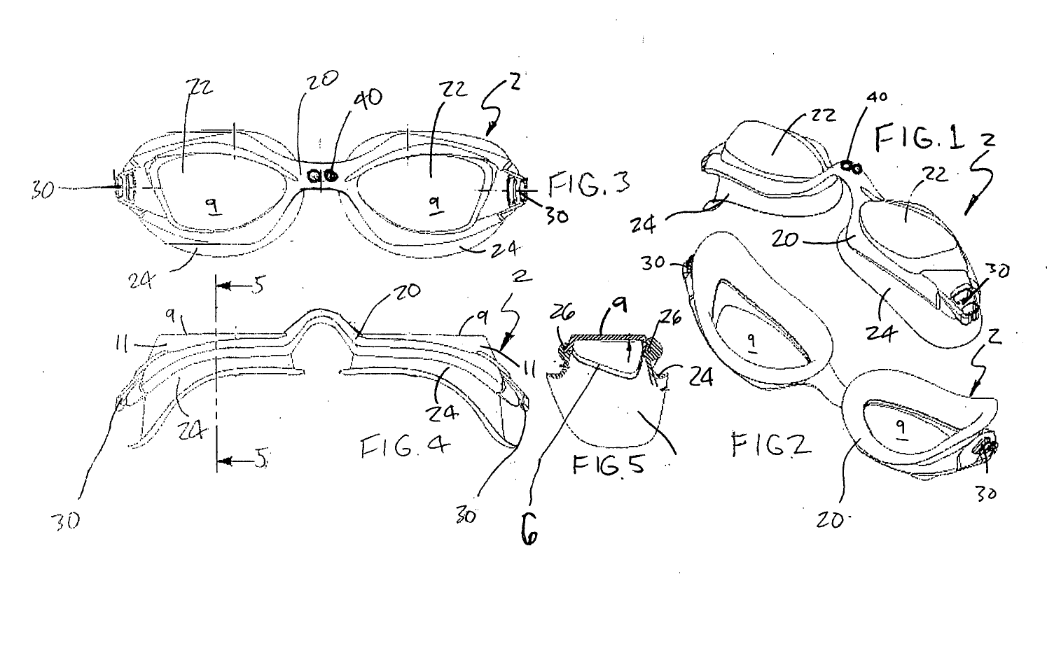

Referring to FIGS. 1 to 4, there is shown a preferred embodiment of eye goggles 2 according to the present invention. The illustrated embodiment relates specifically to goggles intended for swimming, however, the eye goggles of the present invention are not limited to swimming goggles, and swimming goggles are described only by way of example. The structure of the eye goggles of the present invention can be used in goggles intended for various activities where the goal is to protect or isolate a wearer's eyes to prevent water, chemicals or other foreign materials from entering the eyes and obscuring vision.

As best shown in FIGS. 6-8, the eye goggles 2 of the present invention incorporate an inner skeleton 4 of a rigid material. The inner skeleton comprises a pair of transparent eye piece elements 6 joined together by a nose bridge element 8 that is formed integrally with the eye piece elements. Eye piece elements 6 are of conventional shape and dimensions, and spaced apart by nose bridge element 8 to fit over the eyes of a wearer. In the illustrated embodiment, each eye piece element 6 comprises a generally, planar front lens surface 9 with side walls 11 extending rearwardly about the circumference of the lens surface to define a rigid, protective cover positionable over the eye socket of a wearer. Preferably, inner skeleton 4 is all formed from the same transparent material, for example, from an injection moulded polycarbonate plastic to create a self supporting structure. Polycarbonate plastic is preferred as it is clear, strong and lightweight, however, other transparent or translucent plastics can be used. Many current goggle designs use mirror coated, tinted or coloured lenses to reduce glare. Inner skeleton 4 can also be formed from a tinted or coloured polycarbonate or other suitable rigid material.

While nose bridge element 8 is formed integrally with eye piece element 6, the nosebridge element is interconnected between the eye piece elements to accommodate relative movement between the various elements to allow for forming about the nose and eye sockets of a wearer. Preferably, nosebridge element 8 is connected to each eye piece element by a hinge member 10 to permit flexing. Hinge elements 10 are preferably live hinges formed by creating a thin section of material that is able to flex or fold.

To create the novel eye goggle construction of the present invention, inner skeleton 4 shown in FIG. 6-8 is partially covered by a covering 20 to wrap the majority of the skeleton and leave at least a pair of transparent regions 22 in the eye piece elements through which a wearer can see. This finished eye goggle construction with applied covering 20 is shown in FIGS. 1-5.

Preferably, covering 20 is resilient and formed from an elastomer which is overmoulded onto the inner skeleton 4 to bond with the inner skeleton. The overmoulding process can be accomplished using conventional injection or compression moulding techniques or using a conventional casting process.

The applied elastomer is soft and flexible and presents a comfortable surface for contact with the skin of the wearer. The covering covers most the of the inner skeleton except over the front lens surface 9 and the side walls 11 of the eye goggle pieces 6 to define windows through which a wearer can see through the clear or tinted hard plastic of the eye goggle pieces. The covering is preferably opaque to prevent light from entering the eye pieces except through the exposed surfaces 9 and 11 of the eye piece elements 6. Covering material may be selected from materials such as a thermoplastic elastomer, silicone or polyurethane.

An embodiment of the present invention with a particularly distinctive appearance uses an inner skeleton 4 of a tinted or coloured polycarbonate material overmoulded with a non-opaque (i.e. translucent or transparent) resilient covering such that the inner skeleton would be visible through the covering.

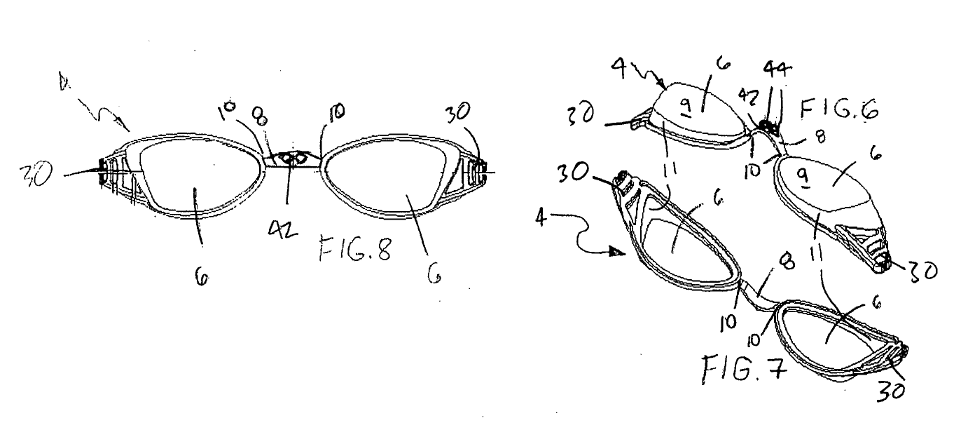

Preferably, covering 20 is formed into a flange 24 extending rearwardly about each rigid eye piece element 6 to define a soft, resilient cup for sealably fitting over a wearer's eye. FIG. 5 shows a cross-section taken along line 5-5 of FIG. 4 in which each eye piece element 6 is formed with a peripheral rim 26 adapted to retain and anchor the inner edge of flange 24.

An important feature of covering 20 is that it extends over the nosebridge piece 8 and hinge members 11 of the inner skeleton 4 to reinforce and support the hinge members to increase the life of the hinges.

In order to reliably attach the eye goggles to the head of a wearer, each eye piece element 6 is preferably formed with a mounting point 30 to receive and retain the end of a strap (not shown) adapted to fit about the head of a user. Mounting point 30 can be of conventional design and is preferably moulded into each eye piece element at a location on each eye piece generally opposite from the nosebridge piece. In the illustrated embodiment, mounting location 30 comprises a slotted structure of conventional design through which the end of a strap is woven. When moulded into eye piece element 6, the slotted structure is left exposed after application of the covering. Other mounting point arrangements known to a person skilled in the art are also possible including attachment of a separate mounting point structure either before applying the overmoulded cover 20 in which case, the mounting point is attached to the eye piece element or after applying the overmoulded cover 20 in which case, the mounting point is attached to or formed in the cover.

The construction of the present invention provides an eye goggle that maintains a desired pre-shaped configuration by virtue of its rigid inner skeleton for fitting over a wearer's eyes. While the eye goggles are sufficiently rigid to retain a desired extended shape for ease of fitting over the face and eyes, they are also comfortable due to the soft, overmoulded covering. The goggles are also constructed from only two main component parts with the result that the overall structure is stronger and assembly costs are significantly reduced.

A unique feature of the goggles of the present invention is the ability to incorporate highly visible indicia on the goggles. Current goggle designs often rely on silkscreened or painted advertising logos or decals to mark the goggles with manufacturer's trademarks. The application of indicia is time consuming and costly, and represents an additional step in the manufacturing process, In addition, indicia marked on conventional goggles are prone to wearing off during normal use. However, marking products with trademark indicia is considered an important step by manufacturer's who wish to distinguish their goggle product from those of competitors.

Establishment of a contrast between the inner skeleton and the outer covering structure of the goggles of the present invention allow for creation of conspicuous, persistent and inexpensive indicia for trademark logos and the like. It is possible to form openings in the covering at the time that the covering 20 is overmoulded onto inner skeleton 4 that define indicia. In many cases, the covering will be opaque over a transparent inner skeleton. The openings in the opaque covering expose regions of the underlying inner skeleton through which light can shine to illuminate the indicia in a clear and eye-catching manner. If direct illumination does not occur, the contrast between the darker outer covering and the lighter indicia will tend to make the indicia stand out conspicuously. Openings 41 in the covering are formed aligned on opposite sides of the transparent inner skeleton to permit light to shine through. At least some of the exposed regions in the inner skeleton may comprise protrusions formed on the inner skeleton that project through the openings in the covering. The openings can define indicia such as abstract logos, lettering or any mark

Referring to FIGS. 3, there are shown exemplary indicia 40 on the goggle nosebridge as described above. In the indicia of FIG. 3, the exposed regions of the inner skeleton comprise the ends 44 of a pair of generally cylindrical protrusions 42 (best shown in FIG. 6) formed on the nose bridge of the inner skeleton to protrude through the covering. The exposed ends 44 of protrusions 42 define a pair of circles as indicia. It will be appreciated that any suitable indicia can be created including abstract shapes, lettering or symbols.

In the case of goggles that employ a non-opaque covering over a tinted or coloured inner skeleton, it is the contrast between the inner skeleton and the outer covering that makes the indicia stand out.

While indicia 40 are shown on the nosebridge portion of the goggles, it is understood that the indicia can be formed on any part of the goggles where covering 20 overlies inner skeleton 4 by formed of appropriate openings in the covering.

Constructing and assembly the eye goggles of the present invention involves forming the inner skeleton of a rigid, polycarbonate plastic to create a pre-shaped skeleton that includes conventional eye pieces joined by a nose bridge element and hinges to permit relative movement between the eye pieces and the nosebridge element. The inner skeleton is then overmoulded with a soft, resilient covering of thermoplastic elastomer, silicone, polyurethane or the like to partially cover the skeleton. The overmoulding step is performed using a conventional process such as injection moulding, compression moulding or casting. Regions of the eye piece elements are left uncovered to define transparent windows through which a wearer can see. When forming the soft, resilient covering over the inner skeleton, it is preferable that eye cup flanges are formed about each eye piece element from the same soft elastomer material for sealably fitting over a wearer's eye.

Forming the covering over the inner skeleton also provides an opportunity for forming the unique indicia mentioned above and illustrated particularly in FIG. 3. By virtue of the inner skeleton being transparent and the covering being opaque or vice versa, indicia can be created in the goggles of the present invention by leaving openings through the covering that expose regions of the inner skeleton. Light shining through the openings serves to illuminate the indicia or the contrast between the inner skeleton visible through the openings and the covering make the indicia stand out.

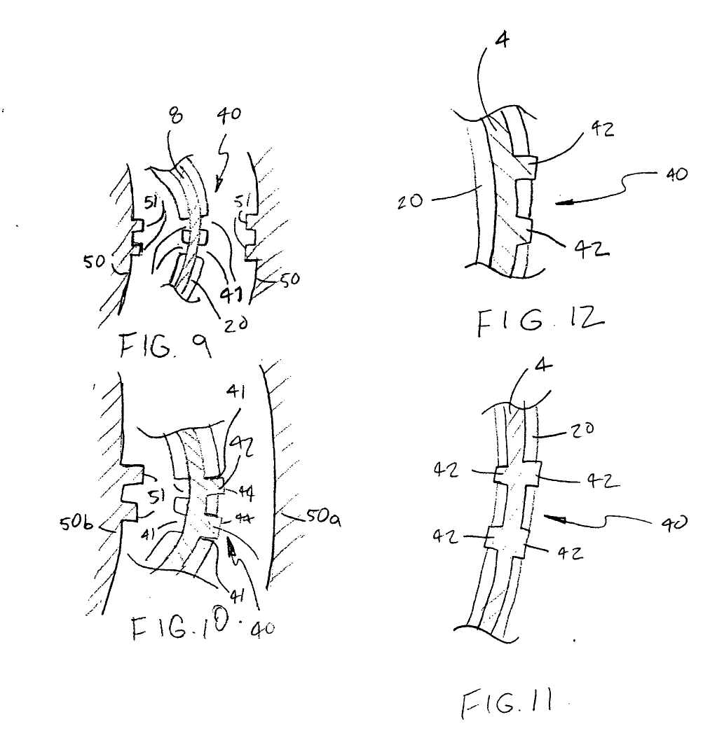

The nose bridge element is a particularly convenient and prominent location on which to form the indicia. For example, during the overmoulding step using an injection moulding process, the inner skeleton is surrounded by opposing mould parts to shape the covering material over the inner skeleton as the material is injected into the mould. Shut off areas are formed on the nose bridge element by the moulds to create appropriate openings in the covering to define the indicia. Examples of separable mould parts 50 configured for forming indicia 40 are shown schematically in FIGS. 9 and 10. These figures show details of a nose bridge element 8 in cross-section after application of the overmoulded covering 20 and partial removal of mould parts 50.

In FIG. 9, nose bridge element 8 is formed without protrusions that define the indicia. In this case, mould parts 50 are formed with projecting fingers 51 that engage the nose bridge element from either side to define shut off areas that prevent the overmoulded material from forming over the nose bridge in the vicinity of the fingers. Finger 51 thereby create aligned openings 41 in the covering on opposite sides of the nose bridge that define the indicia.

As discussed above, the nose bridge element can also be formed with protrusions 42 that protrude from the nose bridge element 8. This arrangement is shown in FIG. 10. In this case, one mould part 50a is adapted to engage the ends 44 of the protrusions 42 on one side of the nose bridge element, while the other mould part 50b includes fingers 51 that define shut off areas to create openings 41 in the covering 20 on the opposite side aligned with and corresponding to the protrusions 42.

While aligned openings 41 are shown in conjunction with protrusions 42 to create indicia in FIG. 10, it is also possible to rely on protrusions 42 alone extending from both sides of the inner skeleton 4 (FIG. 11) or just on one side of the skeleton (FIG. 12) to define the indicia 40.

The arrangements of FIGS. 9, 10 and 11, when used in conjunction with a translucent or transparent inner skeleton 4 and an opaque covering 20, tend to allow light to enter through openings in one side for internal reflection within the skeleton material to illuminate and render conspicuous the indicia at the other side.

In the arrangement of FIG. 12, where protrusions 42 from skeleton 4 are formed on only one side of the skeleton without aligned openings on the opposite side, it is the contrast between the lighter inner skeleton and the darker covering (or vice versa) that make the indicia conspicuous.

Although the present invention has been described in some detail by way of example for purposes of clarity and understanding, it will be apparent that certain changes and modifications may be practised within the scope of the appended claims.

I claim:

1. Eye goggles comprising:
an inner skeleton of a rigid material comprising:
a pair of transparent eye piece elements and
a nose bridge element formed integrally with the eye piece elements and connected therebetween by hinge members to permit relative movement between the eye piece elements and the nose bridge element; and
a covering partially covering the inner skeleton to define at least a pair of transparent regions in the eye piece elements.

2. Eye goggles as claimed in claim 1 in which the inner skeleton is formed from a polycarbonate material.

3. Eye goggles as claimed in claim 1 in which the inner skeleton is formed from a transparent material.

4. Eye goggles as claimed in claim 1 in which the inner skeleton is formed from a translucent material.

5. Eye goggles as claimed in claim 1 in which the hinge members are live hinges.

6. Eye goggles as claimed in claim 1 in which the covering is a resilient covering formed from an elastomer overmoulded onto the inner skeleton.

7. Eye goggles as claimed in claim 6 in which the elastomer is selected from the group consisting of a thermoplastic elastomer, silicone and polyurethane.

8. Eye goggles as claimed in claim 1 in which the covering is formed into a flange extending about each eye piece element to define a cup for sealably fitting over a wearer's eye.

9. Eye goggles as claimed in claim 8 in which each eye piece element includes a peripheral rim adapted to retain an edge of the flange.

10. Eye goggles as claimed in claim 1 in which eye piece element is formed with a mounting point to receive and retain the end of a strap adapted to fit about the head of a user.

11. Eye goggles as claimed in claim 10 in which the mounting point for the head strap is formed at a location on each eye piece generally opposite from the nose bridge element.

12. Eye goggles as claimed in claim 1 in which the covering extends over the nose bridge element and the hinge members of the inner skeleton to reinforce and support the hinge members.

13. Eye goggles as claimed in claim 1 in which the covering is resilient and opaque.

14. Eye goggles as claimed in claim 1 in which the covering is resilient and non-opaque.

15. Eye goggles as claimed in claim 1 including indicia formed on goggles.

16. Eye goggles as claimed in claim 15 in which inner skeleton is formed from one of a non-opaque material and an opaque material and the covering is formed from the other of the non-opaque material and the opaque material and the indicia are defined by regions of the inner skeleton exposed by openings through the covering.

17. Eye goggles as claimed in claim 16 in which at least some of the regions comprise protrusions formed on the inner skeleton that protrude through the covering.

18. Eye goggles as claimed in claim 15 in which the indicia are formed on the nose bridge element.

19. Eye goggles comprising:
an inner skeleton of a rigid material comprising:
a pair of transparent eye piece elements; and
a nose bridge element formed integrally with the eye piece elements and connected therebetween to permit relative movement between the eye piece elements and the nose bridge element; and
a covering partially covering the inner skeleton to define at least a pair of transparent regions in the eye piece elements.

20. Eye goggles as claimed in claim 19 in which the nose piece element is connected to the eye piece elements by hinge members.

21. Eye goggles as claimed in claim 20 in which the hinge members are live hinges.

22. A method of forming eye goggles comprising the steps of:
forming an inner skeleton of a rigid material having a pair of transparent eye piece elements and a nose bridge element formed integrally with the eye piece elements, and connected therebetween to permit relative movement between the eye piece elements and the nose bridge element; and
applying a covering partially covering the inner skeleton to leave at least a pair of transparent regions in the eye piece elements.

23. The method of claim 22 in which the forming step includes the step of forming the inner skeleton from a polycarbonate material.

24. The method of claim 22 in which the forming step includes the step of forming hinge members between the nose bridge element and the eye piece elements.

25. The method of claim 24 in which the step of forming hinge members comprising forming live hinges.

26. The method of claim 24 in which the applying step includes extending the covering over the nose bridge element and the hinge members of the inner skeleton to reinforce and support the hinge members.

27. The method of claim 22 in which the applying step comprising moulding a resilient covering over portions of the inner skeleton.

28. The method of claim 22 in which the applying step comprises forming a flange extending about each eye piece element to define a cup for sealably fitting over a wearer's eye.

29. The method of claim 22 in which the forming step includes forming a mounting point on each eye piece element to receive and retain the ends of a strap adapted to fit about the head of a user.

30. The method of claim 22 in which the applying step comprise moulding a resilient and opaque covering over portions of the inner skeleton.

31. The method of claim 22 including the step of forming indicia on the goggles.

32. The method of claim 31 in which the step of forming indicia involves selecting one of a non-opaque material and an opaque material for the inner skeleton and the other of the non-opaque material and the opaque material for the covering and creating openings through the covering that expose regions of the inner skeleton to define indicia.

33. The method of claim 32 in which the at least some of the regions comprise protrusions formed on the inner skeleton that protrude through the openings in the covering.

34. The method of claim 32 in which the indicia are formed on the nose bridge element.

35. Indicia for eye goggles, said goggles having an inner skeleton enclosed within an outer covering, and said indicia comprising at least one opening in the outer covering to expose a region of the inner skeleton through the covering.

36. Indicia as claimed in claim 35 in which the exposed region of the inner skeleton comprise a protrusion formed on the inner skeleton that protrudes through the opening in the covering.

37. Indicia as claimed in claim 35 in which the inner skeleton is formed from one of a non-opaque material and an opaque material and the outer covering is formed from the other of the non-opaque material and the opaque material.

38. Indicia as claimed in claim 37 in which the inner skeleton is non-opaque and the outer covering is opaque and the at least one opening comprises a pair of aligned openings on opposite sides of the inner skeleton to allow light to pass through the openings and the exposed region of the non-opaque inner skeleton to illuminate the indicia.

39. Indicia as claimed in claim 35 in which the inner skeleton is formed from a rigid material and the outer covering is formed from a resilient material overmoulded over the rigid material.
